(19) United States
(12) Patent Application Publication
(10) Pub. No.: US 2003/0216731 A1
(43) Pub. Date: Nov. 20, 2003

(54) Device and method for facilitating a laparascopic supracervical hysterectomy

(76) Inventor: Martin E. Dennis, Minot, ND (US)

Correspondence Address: TIMOTHY E SIEGEL, 1868 KNAPPS ALLEY, SUITE 206, WEST LINN, OR, 97068, US

(21) Appl. No.: 10/147,617

(22) Filed: May 16, 2002

(51) Int. Cl. A61B 18/18

(52) U.S. Cl. 606/47

(57) ABSTRACT

A method of severing the uterine body from the cervix during the performance of a laparascopic supracervical hysterectomy. A laparascopic instrument having an expandable and contractible thin wire loop at its distal end is provided. The operation starts with the opening of at least one port into the abdomen of the patient. The distal end of the instrument is passed through the port and into the abdominal cavity and the thin wire loop is expanded and passed over the top of the uterus. The thin wire loop is positioned at the boundary between the cervix and the body of the uterus and contracted together, to sever the body of the uterus from the cervix.

### BACKGROUND OF THE INVENTION

In a laparascopic supracervical hysterectomy, the body of the uterus must be detached from the cervix. Presently this is done by using a bipolar needle electrode, which must be inserted through a laparascopic port and manipulated at a distance to repeatedly detach a portion of the cervix a time. This takes between 5 and 10 minutes and may result in damage to neighboring tissue.

The process of unipolar cautery is currently used to, among other functions, removing polyps from the intestines. A number of endoscopic devices made for this purpose have loops that may be pushed outwardly from a lumen, thereby expanding outwardly about the polyp. The loop is then withdrawn into the lumen, causing the portion remaining outside the lumen to contract. Unfortunately, it appears that none of the currently available devices are adaptable for severing the cervix from the uterine body. This is because the uterine body is typically far larger than an intestinal polyp, measuring approximately 5 cm high, from 4-5 cm from back to front and from 5-6 cm from side to side.

U.S. Pat. No. 6,176,858 discloses a device and method for separating the uterine body from the cervix during a laparascopic supracervical hysterectomy. Unfortunately, this device is needlessly complex and its use is quite time consuming. A wire segment must be placed around the cervix inside the body. Then one end of the wire segment must be mated to the other (fasteners are provided) before the loop is pulled to remove the cervix from the uterine body. As the loop cannot be cinched, the cutting operation requires that the wire loop must all be pulled forward, thereby potentially contacting other tissue.

### SUMMARY

In a first separate aspect, the present invention is a method of severing the uterine body from the cervix during the performance of a laparascopic supracervical hysterectomy. A laparascopic instrument having an expandable and contractible thin wire loop at its distal end is provided. The operation starts with the opening of at least one port into the abdomen of the patient. The distal end of the instrument is passed through the port and into the abdominal cavity and the thin wire loop is expanded and passed over the top of the uterus. The thin wire loop is positioned at the boundary between the cervix and the body of the uterus and contracted together, to sever the body of the uterus from the cervix.

In a second separate aspect, the present invention is a laparascopic instrument having a proximal and a distal end and adapted to sever the body of the uterus from the cervix during a laparascopic supracervical hysterectomy. The instrument includes a shaft sized to fit through a laparascopic port and having a proximal and a distal end. In addition, an expandable and contractible unipolar cauterizing loop is positioned to controllably emerge from the distal end of the shaft and is sized to expand sufficiently to fit over the top of the human uterus. This loop is adapted to be cinched together, to cut through the tissue connecting the uterine body to the cervix

The foregoing and other objectives, features and advantages of the invention will be more readily understood upon consideration of the following detailed description of the invention, taken in conjunction with the accompanying drawings.

### BRIEF DESCRIPTION OF THE DRAWINGS

FIG. 1 is a perspective view of a laparascopic instrument designed to facilitate the severing of the uterine body from the cervix during a laparascopic supracervical hysterectomy.

FIG. 2 is a perspective view of the distal end of the laparascopic instrument of FIG. 1, in use during a laparascopic supracervical hysterectomy.

FIG. 3 is an alternative embodiment of the distal end of the laparascopic instrument of claim 1.

FIG. 4 is a perspective view of the laparascopic instrument of FIG. 1, in use during a laparascopic supracervical hysterectomy, showing the wire loop being passed over the top of the uterus.

FIG. 5. is a perspective view of the laparascopic instrument of FIG. 1, in use during a laparascopic supracervical hysterectomy, showing the wire loop cinched up at the boundary between the uterine body and the uterine cervix.

FIG. 6 is a perspective view of the laparascopic instrument of FIG. 1, in use during a laparascopic supracervical hysterectomy, showing the uterine body partially severed from the cervix by the wire loop.

FIG. 7 is a perspective view of the uterine body severed from the cervix.

### DETAILED DESCRIPTION OF THE PREFERRED EMBODIMENT

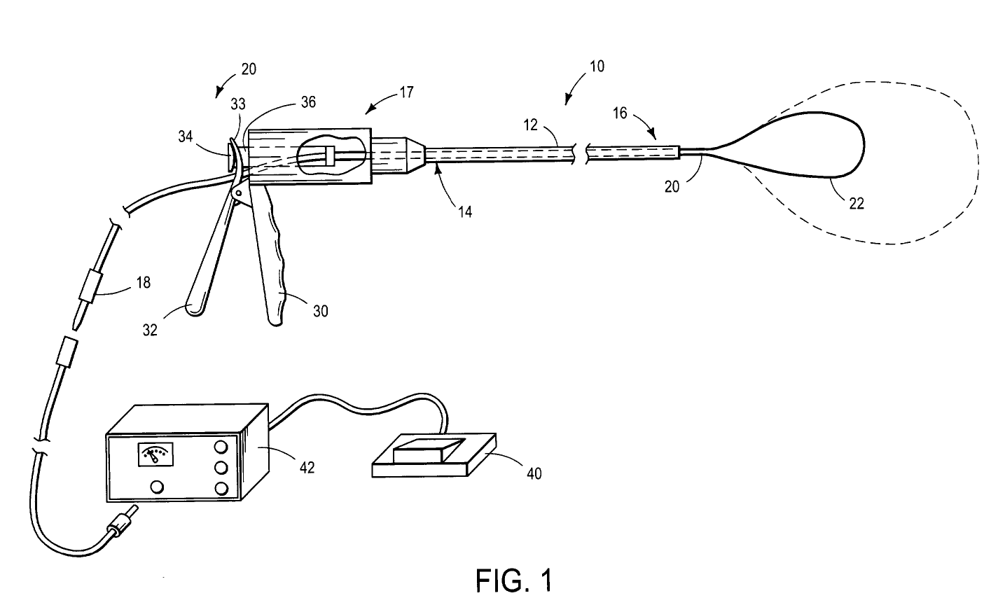

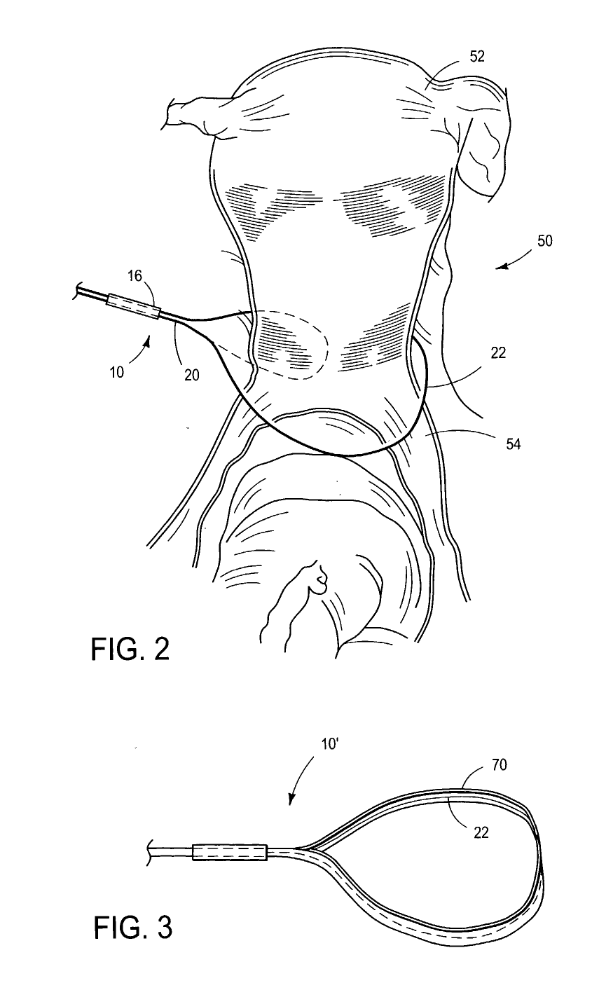

Referring to FIG. 1, a preferred embodiment of a laparascopic instrument 10 according to the present invention has a hollow shaft 12, having a proximal end 14 and a distal end 16. Moreover, shaft 12 defines a lumen and is 30 cm (1 ft) long and has an outer diameter of 1 cm (0.393 in), to fit through a standard laparascopic port. Proximal end 14 expands radially into a handle 17, adapted to be held and manipulated by a physician. A standard electrical jack 18 adapted to connect with a voltage source protrudes from handle 17.

A fine wire 20 is formed into a loop 22, which expands when pushed out of distal end 16 and contracts when withdrawn into distal end 16. A manual actuator 28, including a finger rest 30 and a palm lever 32 having a curved extension 33 that moves an ear 34 of a post 36 that is spring biased to push toward distal end 16. In addition, post 36 is attached to wire 20. This permits the operating physician to push loop 22 out of shaft 12 and to withdraw loop 22 back into shaft 12. A foot pedal 40 is provided for electrical actuation of loop 22 by way of a voltage supply 42.

During an LSH, a pair of laparascopic ports is opened: An upper port directly below the navel and a lower port directly above the pubis. The upper port may be used for illumination, imaging and for some of the preliminary steps of severing the uterus from surrounding tissue. Laparascopic instrument 10 is then introduced through the lower port and loop 22 is expanded into the abdominal cavity, by easing out on palm lever 32, thereby permitting post 36 to push forward, as loop 22 is brought over the top of the uterus 50. Loop 22 is then brought down to the boundary of the uterine body 52 and the cervix 54 and is cinched about the uterus, by pushing on palm lever 32 until some resistance is encountered.

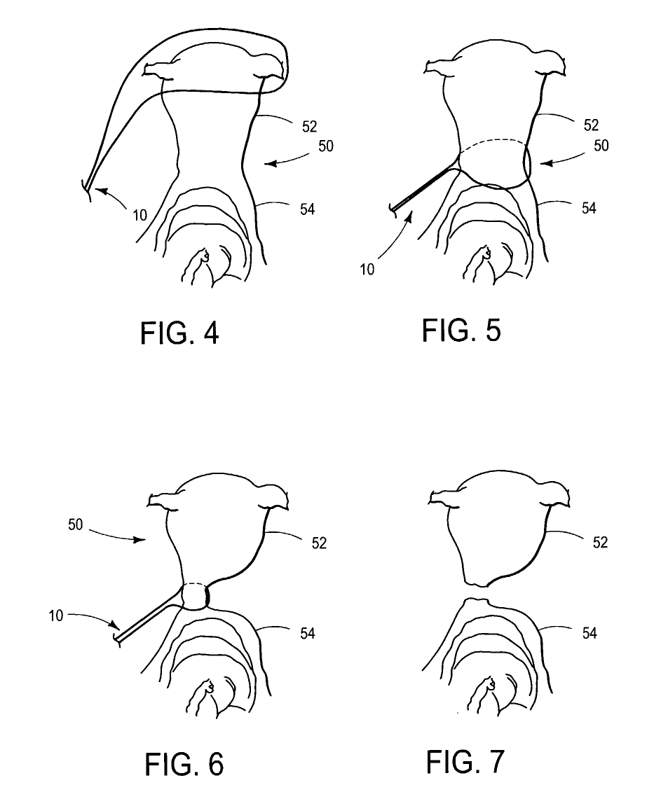

At this point the patient should already be properly grounded, typically with a grounding pad on the thigh. Referring to FIGS. 4 through 7, the loop 22 is then electrically actuated and is further cinched inwardly until the uterine body 52 has been severed from the cervix 54. Instrument 10 is then removed and the operation continues with the morselization of the uterine 52 body, using other instruments.

Referring to FIG. 3, in one preferred embodiment of a laparascopic device 10′, a flexible, dielectric belt 70 is attached to loop 22, so as to shield tissue outside of loop 22 from the effects of the electric current that emanates from loop 22, when it is electrically actuated. In addition, belt 70 provides stiffness to both cause loop 22 to expand outwardly when it is pushed out of shaft 16 and to cause it to better resist the downward force of gravity, as the surgeon attempts to loop it over the top of the uterus 50.

The terms and expressions which have been employed in the foregoing specification are used as terms of description and not of limitation, and there is no intention, in the use of such terms and expressions, of excluding equivalents of the features shown and described or portions thereof, it being recognized that the scope of the invention is defined and limited only by the claims which follow.

1. A method of severing the uterine body from the cervix during the performance of a laparascopic supracervical hysterectomy during which at least one port has been opened into the abdomen of the patient, comprising:
(a) providing a laparascopic instrument having an expandable and contractible thin wire loop at its distal end;
(b) passing said distal end of said instrument through said port and into the abdominal cavity;
(c) expanding said thin wire loop;
(d) passing said thin wire loop over the top of the uterus;
(e) positioning said thin wire loop at the boundary between the cervix and the body of the uterus; and
(f) contracting said thin wire loop together, to sever the body of the uterus from the cervix.

2. The method of claim 1 further including the step of grounding the patient in the general vicinity of the uterus and wherein said thin wire loop is a unipolar cauterizing loop and wherein step (f) includes the electrical activation of said thin wire loop.

3. The method of claim 1 wherein said thin wire loop includes a backing of flexible dielectric material, adapted to provide rigidity to prevent the more distal portion of said expanded loop from being pulled down by gravity.

4. A laparascopic instrument having a proximal and a distal end and adapted to sever the body of the uterus from the cervix during a laparascopic supracervical hysterectomy, said instrument comprising:
(a) a shaft sized to fit through a laparascopic port and having a proximal and a distal end; and
(b) an expandable and contractible unipolar cauterizing loop positioned to controllably emerge from said distal end of said shaft and sized to expand sufficiently to fit over the top of the human uterus and adapted to be cinched together, to cut through the tissue connecting the uterine body to the cervix.

5. The laparascopic instrument of claim 4, wherein said unipolar cauterizing loop includes a backing of flexible dielectric material, adapted to provide rigidity to prevent the more distal portion of said expanded loop from being pulled down by gravity.

6. The instrument of claim 4, further including a manual actuator, positioned at said proximal end of said instrument and adapted to permit expansion and contraction of said loop from said proximal end of said instrument.

7. The instrument of claim 6, wherein said manual actuator permits the full expansion and contraction of said loop within the range of motion of a small human hand.
